(19) United States
(12) Patent Application Publication
(10) Pub. No.: US 2026/0283932 A1
(43) Pub. Date: Sep. 24, 2026

(54) W/O COMPOSITION COMPRISING IONICALLY CROSSLINKED CATIONIC POLYMER SELECTED FROM CHITOSANS AND POLYLYSINES

(71) Applicant: L'OREAL, PARIS (FR)

(72) Inventors: Mariko YAMAMOTO, Kawasaki-shi (JP); Tatsushi ISOJIMA, Kawasaki-shi (JP); Takehiko KASAI, Kawasaki-shi (JP); Kazunori OGAMI, Kawasaki-shi (JP); Mariko OKAMOTO, Kawasaki-shi (JP); Arisa SATO, Kawasaki-shi (JP)

(21) Appl. No.: 19/475,543

(22) PCT Filed: Mar. 29, 2024
(86) PCT No.: PCT/JP2024/080043

§ 371 Date: Oct. 15, 2025

(30) Foreign Application Priority Data
Apr. 18, 2023 (JP) 2023-067934
May 25, 2023 (FR) 2305195

(51) Int. Cl. A61K 8/73; A61K 8/06; A61K 8/55; A61K 8/64; A61K 8/92; A61Q 5/00; A61Q 19/00

(52) U.S. Cl. CPC A61K 8/736; A61K 8/064; A61K 8/55; A61K 8/64; A61K 8/92; A61Q 5/00; A61Q 19/007; A61K 2800/5426

(57) ABSTRACT

A composition contains a plurality of aqueous phases comprising: (a) at least one cationic polymer; (b) at least one non-polymeric acid having two or more pKa values or a salt thereof; and (c) water, and a fatty phase comprising: (d) at least one oil. The aqueous phases are dispersed in the fatty phase, and the (a) cationic polymer is selected from the group consisting of polylysines, chitosans and mixtures thereof, preferably from chitosans. The composition can provide a keratin substance such as skin with, at least, good hydration.

### TECHNICAL FIELD

The present invention relates to a W/O (water-in-oil) composition including an ionically crosslinked cationic polymer selected from chitosans, polylysines and mixtures thereof, as well as a cosmetic process using the composition.

### BACKGROUND ART

As is known in the art, certain cosmetic compositions use a polyion complex, which is formed with an anionic polymer and a cationic polymer.

For example, WO 2021/125069 discloses a composition which is useful for cosmetic treatments and comprises at least one polyion complex particle comprising at least one cationic polymer, at least one anionic polymer and at least one non-polymeric acid having two or more pKa values. WO 2021/125069 also discloses that the composition disclosed therein may include oil and may be in the form of an emulsion.

### DISCLOSURE OF INVENTION

However, the composition disclosed in WO 2021/125069 has been found to be stable only when it includes a very limited amount of oil, such as 0.5% by weight relative to the total weight of the composition. When the composition disclosed therein includes a relatively large amount of oil, it tends to be unstable. Thus, the composition disclosed in WO 2021/125069 is in the form of O/W (oil-in-water).

There is a need for a W/O composition which can provide a keratin substance such as skin with good hydration.

Thus, an objective of the present invention is to provide a W/O composition which can provide a keratin substance such as skin with, at least, good hydration.

The above objective of the present invention can be achieved by a composition, preferably a cosmetic composition, and more preferably a cosmetic composition for a keratin substance such as skin, comprising:
a plurality of aqueous phases comprising:
(a) at least one cationic polymer;
(b) at least one non-polymeric acid having two or more pKa values or a salt thereof; and
(c) water,
and
a fatty phase comprising:
(d) at least one oil,
wherein
the aqueous phases are dispersed in the fatty phase, and
the (a) cationic polymer is selected from the group consisting of chitosans, polylysines, and mixtures thereof, preferably from chitosans.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

The (b) non-polymeric acid having two or more pKa values or a salt thereof may be an organic acid or a salt thereof, preferably a hydrophilic or water-soluble organic acid or a salt thereof, and more preferably phytic acid or a salt thereof.

The amount of the (b) non-polymeric acid(s) having two or more pKa values or salt(s) thereof in the composition according to the present invention may be from 0.001% to 10% by weight, preferably from 0.003% to 5% by weight, and more preferably from 0.005% to 1% by weight, relative to the total weight of the composition.

The amount of the (c) water in the composition according to the present invention may be from 10% to 50% by weight, preferably from 15% to 45% by weight, and more preferably from 20% to 40% by weight, relative to the total weight of the composition.

The amount of the (d) oil(s) in the composition according to the present invention may be from 10% to 50% by weight, preferably from 15% to 45% by weight, and more preferably from 20% to 40% by weight, relative to the total weight of the composition.

The aqueous phase in the composition according to the present invention may further comprise (e) at least one anionic polymer.

The (e) anionic polymer may be selected from the group consisting of polysaccharides such as alginic acid, hyaluronic acid, and cellulose polymers, anionic (co)polyaminoacids such as (co)polyglutamic acids, (co)poly(meth)acrylic acids, (co)polyamic acids, (co)polystyrene sulfonate, (co)poly(vinyl sulfates), dextran sulfate, chondroitin sulfate, (co)polymaleic acids, polyfumaric acids, maleic acid (co)polymers, and salts thereof.

The amount of the (e) anionic polymer(s) in the composition according to the present invention may be from 0.001% to 15% by weight, preferably from 0.005% to 10% by weight, and more preferably from 0.01% to 5% by weight, relative to the total weight of the composition.

The fatty phase in the composition according to the present invention may further comprise (f) at least one fatty acid, preferably selected from C₄-C₂₆, more preferably C₆-C₂₄, and even more preferably C₈-C₂₂ saturated and unsaturated, linear or branched fatty acids.

The amount of the (f) fatty acid(s) in the composition according to the present invention may be from 0.01% to 15% by weight, preferably from 0.05% to 10% by weight, and more preferably from 0.1% to 5% by weight, relative to the total weight of the composition.

The fatty phase in the composition according to the present invention may further comprise (g) at least one organic-modified clay, preferably organic-modified hectorite, and more preferably disteardimonium hectorite.

The amount of the aqueous phases in the composition according to the present invention may be from 30% to 70% by weight, preferably from 35% to 65% by weight, and more preferably from 40% to 60% by weight, relative to the total weight of the composition.

The amount of the fatty phase in the composition according to the present invention may be from 30% to 70% by weight, preferably from 35% to 65% by weight, and more preferably from 40% to 60% by weight, relative to the total weight of the composition.

The present invention also relates to a cosmetic process for a keratin substance such as skin, comprising applying to the keratin substance the composition according to the present invention; and drying the composition to form a cosmetic film on the keratin substance.

### BEST MODE FOR CARRYING OUT THE INVENTION

After diligent research, the inventors have discovered that it is possible to provide a W/O composition which can provide a keratin substance such as skin with, at least, good hydration.

Thus, the composition according to the present invention comprises:
a plurality of aqueous phases comprising:
(a) at least one cationic polymer;
(b) at least one non-polymeric acid having two or more pKa values or a salt thereof; and
(c) water,
and
a fatty phase comprising:
(d) at least one oil,
wherein
the aqueous phases are dispersed in the fatty phase, and
the (a) cationic polymer is selected from the group consisting of chitosans, polylysines, and mixtures thereof, preferably from chitosans.

The (a) cationic polymer can be ionically crosslinked by the (b) non-polymeric acid having two or more pKa values or a salt thereof.

The composition according to the present invention can provide a keratin substance such as skin with good hydration. Thus, the composition according to the present invention can provide a keratin substance such as skin with good moisturizing effects which can be long-lasting.

The composition according to the present invention can also provide a keratin substance such as skin with anti-greasiness and/or anti-color transfer and/or anti-stickiness.

Thus, the composition according to the present invention can provide anti-shine effects which can be long-lasting. Further, the composition according to the present invention can provide anti-color transfer effects, for example, from a face with a makeup such as a foundation to a mask, which can be long-lasting. Furthermore, the composition according to the present invention can provide good texture, such as non-sticky feeling to touch, which can be long-lasting.

If the composition includes (f) at least one fatty acid, the anti-color transfer effects provided by the composition according to the present invention can be further enhanced.

Since chitosans and polylysines can be obtained from natural resources, the (a) cationic polymer is an environmentally-friendly ingredient. Therefore, the composition according to the present invention can include an environmentally-friendly ingredient.

The composition according to the present invention comprises a plurality of aqueous phases, as dispersed or discontinuous phases, comprising the (c) water.

The composition according to the present invention comprises a fatty phase, as a continuous phase, comprising the (d) oil. If the composition according to the present invention comprises (f) at least one fatty acid and/or the (g) at least one organo-modified clay, it or they may be present in the fatty phase.

The (a) cationic polymer can be hydrophobicized by the (f) fatty acid. A part of the hydrophobicized (a) cationic polymer can be present between the fatty phase and the aqueous phases to stabilize the aqueous phases. Thus, the aqueous phases can be stably dispersed in the fatty phase.

The composition according to the present invention is stable for a long period of time. In other words, the phase separation of the composition according to the present invention can be prevented for a long period of time.

Accordingly, the composition according to the present invention can be stored for a long period of time.

The composition according to the present invention can be useful in particular for a foundation or a primer which includes a large amount of a fatty phase, as it can provide long lasting makeup and/or moisturizing effects.

Hereinafter, the present invention will be explained in a more detailed manner.

### [Composition]

### (Cationic Polymer)

The composition according to the present invention comprises (a) at least one cationic polymer.

There is no limit to the type of the (a) cationic polymer. Two or more different types of cationic polymers may be used in combination. Thus, a single type of cationic polymer or a combination of different types of cationic polymers may be used.

A cationic polymer has a positive charge density. The charge density of the (a) cationic polymer may be from 0.01 meq/g to 20 meq/g, preferably from 0.05 to 15 meq/g, and more preferably from 0.1 to 10 meq/g.

The (a) cationic polymer can be included in the aqueous phase including (c) water.

According to the present invention, the (a) cationic polymer is selected from the group consisting of chitosans, polylysines and mixtures thereof.

It is preferable that the (a) cationic polymer be selected from chitosans.

The molecular weight (Da) of the (a) cationic polymer may be less than 20,000, preferably less than 15,000, and more preferably less than 10,000. In other words, the (a) cationic polymer may be a low molecular weight chitosan.

The molecular weight (Da) of the (a) cationic polymer may be more than 1,000, preferably more than 1,500, and more preferably more than 2,000.

Thus, the molecular weight (Da) of the (a) cationic polymer may be more than 1,000 and less than 20,000, preferably more than 1,500 and less than 15,000, and more preferably more than 2,000 and less than 10,000.

Unless otherwise defined in the descriptions, “molecular weight” means a weight average molecular weight. The molecular weight can be measured or determined by a gel permeation chromatography, for example, in accordance with ASTM D5296-19.

Chitosan is very uncommon in nature. It is only reported in the exoskeletons of certain insects such as termite queens and in the cell walls of a particular class of fungi, zygomycetes.

Chitosan may be obtained by deacetylation of chitin. Chitin is a polysaccharide composed of several N-acetyl-D-glucosamine units linked together by a type p bond (1,4).

The ideal chemical structure of chitosan is a sequence of β-D-glucosamine monomers connected by a glycosidic bond (1→4).

“Chitosan” according to the present invention means any copolymer formed of constituent units N-acetyl-D-glucosamine and D-glucosamine, whose degree of acetylation is less than 90%, preferably less than 80%, preferably less than 70%, preferably less than 60%, preferably less than 50%. Chitosan consists of glucosamine sugar units (deacetylated units) and N-acetyl-D-glucosamine units (acetylated units) linked together by β type bonds (1,4) and is a polymer of the Poly (N-acetyl-D-glucosamine)-poly (D-glucosamine) type.

More preferably, the degree of acetylation of chitosan is less than or equal to 40%, preferably less than or equal to 35%, preferably less than or equal to 25%, preferably less than or equal to 15%, and preferably less than or equal to 10%.

The degree of acetylation is the percentage of acetylated units relative to the number of total units, it can be determined by Fourier transform infrared spectroscopy (FT-IR) or by titration by a strong base.

The chitosan of the present invention is preferably a polysaccharide prepared from a fungal origin. In particular, it is extracted and purified from safe and abundant food or biotechnological fungal sources such as Agaricus bisporus or Aspergillus niger.

The chitosan of the present invention is preferably derived from the mycelium of a fungus of the Ascomycete type, and in particular Aspergillus niger and/or a Basidiomycete fungus, and in particular Lentinula edodes (shiitake) and/or Agaricus bisporus. Preferably the fungus is Aspergillus niger.

Chitosan may be of GMO (Genetically Modified Organisms) origin, but preferably is of non-GMO origin.

The chitosan according to the present invention is native, that is to say that it is not modified.

In particular, it does not contain any chemical modification.

One method of preparing chitosan is that described in WO03/068824.

Preferably, the chitosan used in the present invention is in a powder form. It is marketed by Glentham Life Science under the name GU3511.

Polylysine is also well known.

Polylysine can be a natural homopolymer of L-lysine that can be produced by bacterial fermentation. For example, polylysine can be ε-Poly-L-lysine, typically used as a natural preservative in food products. Polylysine is a polyelectrolyte which is soluble in polar solvents such as water, propylene glycol and glycerol. Polylysine is commercially available in various forms, such as poly D-lysine and poly L-lysine. Poly-L-Lysine is preferable. Polylysine can be in salt and/or solution form.

The amount of the (a) cationic polymer(s) in the composition according to the present invention may be 0.01% by weight or more, preferably 0.05% by weight or more, and more preferably 0.1% by weight or more, relative to the total weight of the composition.

The amount of the (a) cationic polymer(s) in the composition according to the present invention may be 15% by weight or less, preferably 10% by weight or less, and more preferably 5% by weight or less, relative to the total weight of the composition.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

### (Non-Polymeric Acid Having Two or More Acid Dissociation Constants)

The composition according to the present invention comprises (b) at least one non-polymeric acid having two or more pKa values or a salt thereof, i.e., at least one non-polymeric acid having two or more acid dissociation constants or a salt thereof. The pKa value (acid dissociation constant) is well known to those skilled in the art, and should be determined at a constant temperature such as 25° C.

The (b) non-polymeric acid having two or more pKa values or a salt thereof can be included in the aqueous phase including (c) water. The non-polymeric acid having two or more pKa values can function as a crosslinker for the (a) cationic polymer.

The term “non-polymeric” here means that the acid is not obtained by polymerizing two or more monomers. Therefore, the non-polymeric acid does not correspond to an acid obtained by polymerizing two or more monomers, such as polyacrylic acid.

It is preferable that the molecular weight of the (b) non-polymeric acid having two or more pKa values or a salt thereof is 1000 or less, preferably 800 or less, and more preferably 700 or less.

There is no limit to the type of the (b) non-polymeric acid having two or more pKa values or a salt thereof. Two or more different types of (b) non-polymeric acids having two or more pKa values or salts thereof may be used in combination. Thus, a single type of a (b) non-polymeric acid having two or more pKa values or a salt thereof or a combination of different types of (b) non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metal such as Na and K, and salts with alkaline earth metal such as Mg and Ca, and ammonium salts.

The non-polymeric acid having two or more pKa values or a salt thereof may be an organic acid or a salt thereof, and preferably a hydrophilic or water-soluble organic acid or a salt thereof.

The non-polymeric acid having two or more pKa values may have at least two acid groups selected from the group consisting of a carboxylic group, a sulfuric group, a sulfonic group, a phosphoric group, a phosphonic group, a phenolic hydroxyl group, and a mixture thereof.

The non-polymeric acid having two or more pKa values may be a non-polymeric polyvalent acid such as phosphoric acid.

The non-polymeric acid having two or more pKa values may be selected from the group consisting of dicarboxylic acids, disulfonic acids, and diphosphoric acids, and a mixture thereof.

The (b) non-polymeric acid having two or more pKa values or a salt thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, aconitic acid, oxaloacetic acid, tartaric acid, and salts thereof; aspartic acid, glutamic acid, and salts thereof; terephthalylidene dicamphor sulfonic acid or salts thereof (Mexoryl SX), Benzophenone-9; phytic acid, and salts thereof; Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Cone), Green 205 (Light Green SF Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphtol Green B), Green 402 (Guinea Green B), and Black 401 (Naphtol Blue Black); folic acid, ascorbic acid, erythorbic acid, and salts thereof; cystine and salts thereof; EDTA and salts thereof; glycyrrhizin and salts thereof; and a mixture thereof.

It may be preferable that the (b) non-polymeric acid having two or more pKa values or a salt thereof be selected from the group consisting of terephthalylidene dicamphor sulfonic acid and salts thereof (Mexoryl SX), Yellow 6 (Sunset Yellow FCF), ascorbic acid, phytic acid and salts thereof, and a mixture thereof.

The (b) non-polymeric acid having two or more pKa values or a salt thereof may be an organic acid or a salt thereof, preferably a hydrophilic or water-soluble organic acid or a salt thereof, and more preferably phytic acid or a salt thereof.

The amount of the (b) non-polymeric acid(s) having two or more pKa values or salt(s) thereof in the composition according to the present invention may be 0.001% by weight or more, preferably 0.003% by weight or more, and more preferably 0.005% by weight or more, relative to the total weight of the composition.

The amount of the (b) non-polymeric acid(s) having two or more pKa values or salt(s) thereof in the composition according to the present invention may be 10% by weight or less, preferably 5% by weight or less, and more preferably 1% by weight or less, relative to the total weight of the composition.

The amount of the (b) non-polymeric acid(s) having two or more pKa values or salt(s) thereof in the composition according to the present invention may be from 0.001% to 10% by weight, preferably from 0.003% to 5% by weight, and more preferably from 0.005% to 1% by weight, relative to the total weight of the composition.

### (Water)

The composition according to the present invention comprises (c) water.

The (c) water can constitute the aqueous phases, which can be dispersed or discontinuous phases, in the composition according to the present invention.

The amount of the (c) water may be 10% by weight or more, preferably 15% by weight or more, and more preferably 20% by weight or more, relative to the total weight of the composition.

The amount of the (c) water may be 50% by weight or less, preferably 45% by weight or less, and more preferably 40% by weight or less, relative to the total weight of the composition.

The amount of the (c) water may be from 10% to 50% by weight, preferably from 15% to 45% by weight, and more preferably from 20% to 40% by weight, relative to the total weight of the composition.

### (Oil)

The composition according to the present invention comprises (d) at least one oil. If two or more (d) oils are used, they may be the same or different.

The (d) oil(s) can constitute a fatty phase, which can be a continuous phase, in the composition according to the present invention.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (d) oil may be a non-polar oil such as a hydrocarbon oil, a silicone oil, or the like; a polar oil such as a plant or animal oil and an ester oil or an ether oil; or a mixture thereof.

The (d) oil may be selected from the group consisting of oils of plant or animal origin, synthetic oils, silicone oils, hydrocarbon oils and fatty alcohols.

As examples of plant oils, mention may be made of, for example, apricot oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) Cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

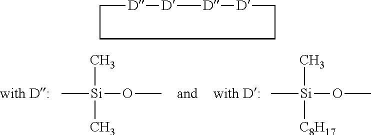

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) Linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil© oils sold by Rhodia, for instance the oil 70 047 V 500 000;
the oils of the Mirasil© series sold by the company Rhodia;
the oils of the 200 series from the company Dow Corning, such as DC200 with a viscosity of 60,000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula

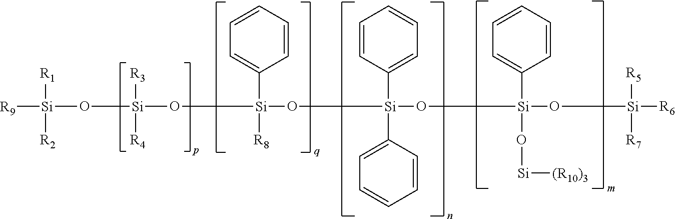

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and
m, n, p and q are, independently of each other, integers 0 to 900 inclusive, preferably 0 to 500 inclusive, and more preferably 0 to 100 inclusive,
with the proviso that the sum n+m+q is not 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

It may be preferable that the (d) oil be selected from synthetic ester oils, hydrocarbon oils, silicone oils, and mixtures thereof.

The amount of the (d) oil(s) in the composition according to the present invention may be 10% by weight or more, preferably 15% by weight or more, and more preferably 20% by weight or more, relative to the total weight of the composition.

The amount of the (d) oil(s) in the composition according to the present invention may be 50% by weight or less, preferably 45% by weight or less, and more preferably 40% by weight or less, relative to the total weight of the composition.

The amount of the (d) oil(s) in the composition according to the present invention may be from 10% to 50% by weight, preferably from 15% to 45% by weight, and more preferably from 20% to 40% by weight, relative to the total weight of the composition.

### (Anionic Polymer)

The composition according to the present invention may comprise (e) at least one anionic polymer. A single type of anionic polymer may be used, or two or more different types of anionic polymers may be used in combination.

An anionic polymer has a negative charge density. The charge density of the (e) anionic polymer may be from 0.1 meq/g to 20 meq/g, preferably from 1 meq/g to 15 meq/g, and more preferably from 4 meq/g to 10 meq/g if the (e) anionic polymer is a synthetic anionic polymer, and the average substitution degree of the (e) anionic polymer may be from 0.1 to 3.0, preferably from 0.2 to 2.7, and more preferably from 0.3 to 2.5 if the (e) anionic polymer is a natural anionic polymer.

It may be preferable that the molecular weight of the (e) anionic polymer be 1,000 or more, preferably 2,000 or more, even more preferably 5,000 or more, even more preferably 10,000 or more, even more preferably 50,000 or more, even more preferably 100,000 or more, and even more preferably 1,000,000 or more.

Unless otherwise defined in the descriptions, “molecular weight” may mean a weight average molecular weight.

The (e) anionic polymer may have at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a carboxylic group, and a carboxylate group.

The (e) anionic polymer may be a homopolymer or a copolymer. The term “copolymer” is understood to mean both copolymers obtained from two kinds of monomers and those obtained from more than two kinds of monomers, such as terpolymers obtained from three kinds of monomers.

The (e) anionic polymer may be selected from natural and synthetic anionic polymers, and preferably from natural anionic polymers.

The (e) anionic polymer may comprise at least one hydrophobic chain.

The (e) anionic polymer which may comprise at least one hydrophobic chain may be obtained by copolymerization of a monomer (a) chosen from carboxylic acids comprising α,β-ethylenic unsaturation (monomer a′) and 2-acrylamido-2-methylpropanesulphonic acid (monomer a″) with a non-surface-active monomer (b) comprising an ethylenic unsaturation other than (a) and/or a monomer (c) comprising an ethylenic unsaturation resulting from the reaction of an acrylic monomer comprising an α,β-monoethylenic unsaturation or of an isocyanate monomer comprising a monoethylenic unsaturation with a monohydric nonionic amphiphilic component or with a primary or secondary fatty amine.

Thus, the (e) anionic polymer with at least one hydrophobic chain may be obtained by two synthetic routes:
either by copolymerization of the monomers (a′) and (c), or (a′), (b) and (c), or (a″) and (c), or (a″), (b) and (c),
or by modification (and in particular esterification or amidation) of a copolymer formed from the monomers (a′) or from the monomers (a′) and (b), or (a″) and (b), by a monohydric nonionic amphiphilic compound or a primary or secondary fatty amine.

Mention may in particular be made, as 2-acrylamido-2-methylpropanesulphonic acid copolymers, of those disclosed in the article “Micelle formation of random copolymers of sodium 2-(acrylamido)-2-methylpropanesulfonate and nonionic surfactant macromonomer in water as studied by fluorescence and dynamic light scattering—Macromolecules, 2000, Vol. 33, No. 10-3694-3704” and in applications EP-A-0 750 899 and EP-A-1 069 172.

The carboxylic acid comprising an α,β-monoethylenic unsaturation constituting the monomer (a′) can be chosen from numerous acids and in particular from acrylic acid, methacrylic acid, crotonic acid, itaconic acid and maleic acid. It is preferably acrylic or methacrylic acid.

The copolymer can comprise a monomer (b) comprising a monoethylenic unsaturation which does not have a surfactant property. The preferred monomers are those which give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The more particularly preferred monomers are methyl acrylate and ethyl acrylate. The other monomers which can be used are, for example, styrene, vinyltoluene, vinyl acetate, acrylonitrile and vinylidene chloride. Unreactive monomers are preferred, these monomers being those in which the single ethylenic group is the only group which is reactive under the polymerization conditions. However, monomers which comprise groups which react under the effect of heat, such as hydroxyethyl acrylate, can optionally be used.

The monomer (c) is obtained by reaction of an acrylic monomer comprising α,β-monoethylenic unsaturation, such as (a), or of an isocyanate monomer comprising monoethylenic unsaturation with a monohydric nonionic amphiphilic compound or a primary or secondary fatty amine.

The monohydric nonionic amphiphilic compounds or the primary or secondary fatty amines used to produce the nonionic monomer (c) are well known. The monohydric nonionic amphiphilic compounds are generally alkoxylated hydrophobic compounds comprising an alkylene oxide forming the hydrophilic part of the molecule. The hydrophobic compounds are generally composed of an aliphatic alcohol or an alkylphenol, in which compounds a carbonaceous chain comprising at least six carbon atoms constitutes the hydrophobic part of the amphiphilic compound.

The preferred monohydric nonionic amphiphilic compounds are compounds having the following formula (V):

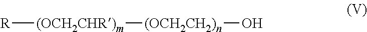

in which R is chosen from alkyl or alkylene groups comprising from 6 to 30 carbon atoms and alkylaryl groups having alkyl radicals comprising from 8 to 30 carbon atoms, R′ is chosen from alkyl groups comprising from 1 to 4 carbon atoms, n is a mean number ranging from approximately 1 to 150 and m is a mean number ranging from approximately 0 to 50, provided that n is at least as great as m.

Preferably, in the compounds of formula (V), the R group is chosen from alkyl groups comprising from 12 to 26 carbon atoms and alkylphenyl groups in which the alkyl group is C₈-C₁₃; the R′ group is the methyl group; m=0 and n=1 to 25.

The preferred primary and secondary fatty amines are composed of one or two alkyl chains comprising from 6 to 30 carbon atoms.

The monomer used to form the nonionic urethane monomer (c) can be chosen from highly varied compounds. Use may be made of any compound comprising a copolymerizable unsaturation, such as an acrylic, methacrylic or allylic unsaturation. The monomer (c) can be obtained in particular from an isocyanate comprising a monoethylenic unsaturation, such as, in particular, α,α-dimethyl-m-isopropenylbenzyl isocyanate.

The monomer (c) can be chosen in particular from acrylates, methacrylates or itaconates of oxyethylenated (1 to 50 EO) C₆-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate or the acrylate modified by polyoxyethylenated (25 EO) C₁₂-C₂₄ alcohols and from dimethyl-m-isopropenylbenzyl isocyanates of oxyethylenated (1 to 50 EO) C₆-C₃₀ fatty alcohol, such as, in particular, the dimethyl-m-isopropenylbenzyl isocyanate of oxyethylenated behenyl alcohol.

According to a specific embodiment of the present invention, the (e) anionic polymer is chosen from acrylic terpolymers obtained from (a) a carboxylic acid comprising an α,β-ethylenic unsaturation, (b) a non-surface-active monomer comprising an ethylenic unsaturation other than (a), and (c) a nonionic urethane monomer which is the reaction product of a monohydric nonionic amphiphilic compound with an isocyanate comprising a monoethylenic unsaturation.

Mention may in particular be made, as the (e) anionic polymers comprising at least one hydrophobic chain, of the acrylic acid/ethyl acrylate/alkyl acrylate terpolymer, such as the product as a 30% aqueous dispersion sold under the name Acusol 823 by Rohm & Haas; the acrylates/steareth-20 methacrylate copolymer, such as the product sold under the name Aculyn 22 by Rohm & Haas; the (meth)acrylic acid/ethyl acrylate/oxyethylenated (25 EO) behenyl methacrylate terpolymer, such as the product as an aqueous emulsion sold under the name Aculyn 28 by Rohm & Haas; the acrylic acid/oxyethylenated (20 EO) monocetyl itaconate copolymer, such as the product as a 30% aqueous dispersion sold under the name Structure 3001 by National Starch; the acrylic acid/oxyethylenated (20 EO) monostearyl itaconate copolymer, such as the product as a 30% aqueous dispersion sold under the name Structure 2001 by National Starch; the acrylates/acrylate modified by polyoxyethylenated (25 EO) C₁₂-C₂₄ alcohol copolymer, such as the 30-32% copolymer latex sold under the name Synthalen W2000 by 3V SA; or the methacrylic acid/methyl acrylate/dimethyl-meta-isopropenylbenzyl isocyanate of ethoxylated behenyl alcohol terpolymer, such as the product as a 24% aqueous dispersion and comprising 40 ethylene oxide groups disclosed in the document EP-A-0 173 109.

The (e) anionic polymers may also be Polyester-5, such as the product sold under the name of Eastman AQ™ 55S Polymer by EASTMAN CHEMICAL having a chemical formula below.

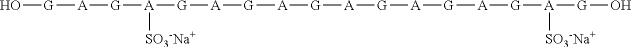

A: dicarboxylic acid moiety
G: glycol moiety
SO₃⁻Na⁺: sodium sulfo group
OH: hydroxyl group

It may be preferable that the (e) anionic polymer be selected from the group consisting of polysaccharides such as alginic acid, hyaluronic acid, xanthan gum, and cellulose polymers (e.g., carboxymethylcellulose), anionic (co)polyaminoacids such as (co)polyglutamic acids, (co)poly(meth)acrylic acids, (co)polyamic acids, (co)polystyrene sulfonate, (co)poly(vinyl sulfate), dextran sulfate, chondroitin sulfate, (co)polymaleic acids, (co)polyfumaric acids, maleic acid (co)polymers, and salts thereof.

The maleic acid copolymer may comprise one or more maleic acid comonomers, and one or more comonomers chosen from vinyl acetate, vinyl alcohol, vinylpyrrolidone, olefins comprising from 2 to 20 carbon atoms, and styrene.

Thus, the “maleic acid copolymer” is understood to mean any polymer obtained by copolymerization of one or more maleic acid comonomers and of one or more comonomers chosen from vinyl acetate, vinyl alcohol, vinylpyrrolidone, olefins comprising from 2 to 20 carbon atoms, such as octadecene, ethylene, isobutylene, diisobutylene or isooctylene, and styrene, the maleic acid comonomers optionally being partially or completely hydrolysed. Use will preferably be made of hydrophilic polymers, that is to say polymers having a solubility of water of greater than or equal to 2 g/l.

In an advantageous aspect of the present invention, the maleic acid copolymer may have a molar fraction of maleic acid units of between 0.1 and 1, more preferably between 0.4 and 0.9.

The weight-average molar mass of the maleic acid copolymer may be between 1,000 and 500,000, and preferably between 1,000 and 50,000.

It is preferable that the maleic acid copolymer be a styrene/maleic acid copolymer, and more preferably sodium styrene/maleic acid copolymer.

Use will preferably be made of a copolymer of styrene and of maleic acid in a 50/50 ratio.

Use may be made, for example, of the styrene/maleic acid (50/50) copolymer, in the form of an ammonium salt at 30% in water, sold under the reference SMA1000H® by Cray Valley or the styrene/maleic acid (50/50) copolymer, in the form of a sodium salt at 40% in water, sold under the reference SMA1000HNa® by Cray Valley.

The use of the styrene/maleic acid copolymer such as sodium styrene/maleic acid copolymer can improve the wettability of a film prepared by the composition according to the present invention.

In a preferable embodiment, the (e) anionic polymer may be selected from hyaluronic acid, salts thereof (e.g., sodium hyaluronate), and derivatives thereof.

Hyaluronic acid can be represented by the following chemical formula.

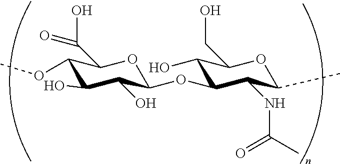

In the context of the present invention, the term “hyaluronic acid” covers in particular the basic unit of hyaluronic acid of formula:

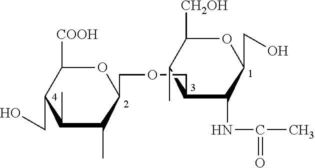

It is the smallest fraction of hyaluronic acid comprising a disaccharide dimer, namely D-glucuronic acid and N-acetylglucosamine.

The term “hyaluronic acid and derivatives thereof” also comprises, in the context of the present invention, the linear polymer comprising the polymeric unit described above, linked together in the chain via alternating β(1,4) and β(1,3) glycosidic linkages, having a molecular weight (MW) that can range between 380 and 13 000 000 daltons. This molecular weight depends in large part on the source from which the hyaluronic acid is obtained and/or on the preparation methods.

The term “hyaluronic acid and derivatives thereof” also comprises, in the context of the present invention, the hyaluronic acid salts. As the salts, mention may be made of alkaline metal salts such as sodium salts and potassium salts, alkaline earth metal salts such as magnesium salts, ammonium salts, and mixtures thereof.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid and derivatives thereof” comprises all the fractions or subunits of hyaluronic acid having a molecular weight in particular within the molecular weight range recalled above.

In the context of the present invention, hyaluronic acid fractions which do not have an inflammatory activity are preferably used.

By way of illustration of the various hyaluronic acid fractions, reference may be made to the document “Hyaluronan fragments: an information-rich system”, R. Stem et al., European Journal of Cell Biology 58 (2006) 699-715, which reviews the listed biological activities of hyaluronic acid according to its molecular weight.

According to a preferred embodiment of the present invention, the hyaluronic acid fractions suitable for the use covered by the present invention have a molecular weight of between 50 000 and 5 000 000, in particular between 100 000 and 5 000 000, especially between 400 000 and 5 000 000 Da. In this case, the term used is high-molecular-weight hyaluronic acid.

Alternatively, the hyaluronic acid fractions that may also be suitable for the use covered by the present invention have a molecular weight of between 50 000 and 400 000 Da. In this case, the term used is intermediate-molecular-weight hyaluronic acid.

Alternatively again, the hyaluronic acid fractions that may be suitable for the use covered by the present invention have a molecular weight of less than 50 000 Da. In this case, the term used is low-molecular-weight hyaluronic acid.

Finally, the term “hyaluronic acid and derivatives thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The hyaluronic acid derivative may be, for example, acetylated hyaluronic acid or a salt thereof.

The molecular weights indicated above are also valid for the hyaluronic acid esters.

Hyaluronic acid may in particular be hyaluronic acid supplied by the company Hyactive under the trade name CPN (MW: 10 to 150 kDa), by the company Soliance under the trade 50 name Cristalhyal (MW: 1.1.times.10⁶), by the company Bioland under the name Nutra HA (MW: 820 000 Da), by the company Bioland under the name Nutra AF (MW: 69 000 Da), by the company Bioland under the name Oligo HA (MW: 6100 Da) or else by the company Vam Farmacos Metica under the name D Factor (MW: 380 Da).

The amount of the (e) anionic polymer(s) in the composition according to the present invention may be 0.001% by weight or more, preferably 0.005% by weight or more, and more preferably 0.01% by weight or more, relative to the total weight of the composition.

The amount of the (e) anionic polymer(s) in the composition according to the present invention may be 15% by weight or less, preferably 10% by weight or less, and more preferably 5% by weight or less, relative to the total weight of the composition.

The amount of the (e) anionic polymer(s) in the composition according to the present invention may be from 0.001% to 15% by weight, preferably from 0.005% to 10% by weight, and more preferably from 0.01% to 5% by weight, relative to the total weight of the composition.

### (Fatty Acid)

The composition according to the present invention may comprise (f) at least one fatty acid.

If two or more fatty acids are used, they may be the same or different.

The term “fatty acid” here means a carboxylic acid with a long aliphatic carbon chain.

The (f) fatty acid has at least 4 carbon atoms, preferably at least 6 carbon atoms, and more preferably at least 8 carbon atoms. The (f) fatty acid may comprise up to 26 carbon atoms, preferably up to 24 carbon atoms, and more preferably up to 22 carbon atoms. It is preferable that the (f) fatty acid be selected from C₄-C₂₆ fatty acid, more preferably C₆-C₂₄ fatty acid, and even more preferably C₈-C₂₂ fatty acid.

The (f) fatty acid may be selected from saturated or unsaturated, linear or branched fatty acids. Thus, the (f) fatty acid may be selected from C₄-C₂₆, preferably C₆-C₂₄, more preferably C₈-C₂₂ saturated and unsaturated, linear or branched fatty acids.

As the unsaturated, linear or branched fatty acids, mono-unsaturated, linear or branched fatty acids or polyunsaturated, linear or branched fatty acids may be used. As the unsaturated moiety of the unsaturated, linear or branched fatty acids, a carbon-carbon double bond or a carbon-carbon triple bond may be mentioned.

As the saturated fatty acid, mention may be made of, for example, caprylic acid (C₈), pelargonic acid (C₉), capric acid (C₁₀), lauric acid (C₁₂), myristic acid (C₁₄), pentadecanoic acid (C₁₅), palmitic acid (C₁₆), heptadecanoic acid (C₁₇), stearic acid (C₁₈), isostearic acid (C₁₈), nonadecanoic acid (C₁₉), arachidic acid (C₂₀), behenic acid (C₂₂), and lignoceric acid (C₂₄).

As the unsaturated fatty acid, mention may be made of, for example, myristoleic acid (C₁₄), palmitoleic acid (C₁₆), oleic acid (C₁₈), linoleic acid (C₁₈), linolenic acid (C₁₈), elaidic acid (C₁₈), arachidonic acid (C₂₀), eicosenoic acid (C₂₀), erucie acid (C₂₂), and nervonic acid (C₂₄).

It is preferable that the (f) fatty acid be selected from C₈-C₁₈ saturated or unsaturated, linear or branched fatty acids, and more preferably from the group consisting of caprylic acid, capric acid, oleic acid, linoleic acid, stearic acid, isostearic acid and mixtures thereof.

The (f) fatty acid may be in the form of a free acid or in the form of a salt thereof. As a salt of the fatty acid, mention may be made of an inorganic salt such as an alkali metal salt (a sodium salt, a potassium salt, or the like) and an alkaline earth metal salt (a magnesium salt, a calcium salt, or the like); and an organic salt such as an ammonium salt (a quaternary ammonium salt or the like) and an amine salt (a triethanolamine salt, a triethylamine salt, or the like). A single type of fatty acid salt or a combination of different type of fatty acid salts may be used. Further, a combination of one or more fatty acid in the form of a free acid and one or more fatty acid in the form of a salt may be used, in which one or more type of salts may also be used.

The amount of the (f) fatty acid(s) in the composition according to the present invention may be 0.01% by weight or more, preferably 0.05% by weight or more, and more preferably 0.1% by weight or more, relative to the total weight of the composition. It may be even more preferable that the amount of the (f) fatty acid(s) in the composition according to the present invention be 1% by weight or more, relative to the total weight of the composition.

On the other hand, the amount of the (f) fatty acid(s) in the composition according to the present invention may be 15% by weight or less, preferably 10% by weight or less, and more preferably 5% by weight or less, relative to the total weight of the composition. It may be even more preferable that the amount of the (f) fatty acid(s) in the composition according to the present invention be 4% by weight or less, relative to the total weight of the composition.

Accordingly, the amount of the (f) fatty acid(s) in the composition according to the present invention may range from 0.01% to 15% by weight, preferably from 0.05% to 10% by weight, and more preferably from 0.1% to 5% by weight, relative to the total weight of the composition. It may be even more preferable that the amount of the (f) fatty acid(s) in the composition according to the present invention be from 1% to 4% by weight, relative to the total weight of the composition.

### (Organo-Modified Clay)

The composition according to the present invention may comprise (g) at least one organo-modified clay. If two or more organo-modified clays are used, they may be the same or different.

The organo-modified clay means clays treated with organic compounds, especially selected from quaternary amines and tertiary amines. By exchanging the original interlayer cations for organocations (typically quaternary or tertiary alkylammonium ions), an organophilic surface is generated, comprising covalently linked organic moieties.

The (g) organo-modified clay may be present between the fatty phase and the aqueous phase to stabilize the aqueous phase in the composition according to the present invention.

It may be preferable that the (g) organomodified clay be in the form of particles.

Organo-modified clays that may be mentioned include organo-modified bentonites and hectorites, such as the product sold under the name Bentone 34 by the company Rheox, and organo-modified hectorites such as the products sold under the names Bentone 27 (stearalkonium hectorite) and Bentone 38 (disteardimonium hectorite) by the company Rheox, and the name MP250 (stearalkonium bentonite) by the company BYK Additives & Instrumentals.

Mention may be made especially of modified clays such as modified magnesium silicate (Bentone gel VS38 from Rheox), modified hectorites such as hectorite modified with a C10 to C22 fatty acid ammonium chloride, for instance hectorite modified with distearyldimethylammonium chloride, for instance the product sold under the name Bentone 38VCG by the company Elementis or the product sold under the name Bentone 38 CE by the company Rheox, or the product sold under the name Bentone Gel V55V by the company Elementis.

It is preferable that the (g) organo-modified clay be chosen from organo-modified bentonites, organo-modified hectorites and mixtures thereof. As the (g) organo-modified clay, mention may be made of disteardimonium hectorite.

It is preferable that the (g) organo-modified clay has been treated with compounds chosen especially from quaternary amines and tertiary amines.

The amount of the (g) organo-modified clay(s) in the composition according to the present invention may be 0.01% by weight or more, preferably 0.05% by weight or more, and more preferably 0.1% by weight or more, relative to the total weight of the composition.

The amount of the (g) organo-modified clay(s) in the composition according to the present invention may be 15% by weight or less, preferably 10% by weight or less, and more preferably 5% by weight or less, relative to the total weight of the composition.

The amount of the (g) organo-modified clay(s) in the composition according to the present invention may be from 0.01% to 15% by weight, preferably from 0.05% to 10% by weight, and more preferably from 0.1% to 5% by weight, relative to the total weight of the composition.

(pH)

The pH of the composition according to the present invention may be from 3 to 9, preferably from 3.3 to 8.5, and more preferably from 3.5 to 8.

At a pH of from 3 to 9, the (a) cationic polymer or a complex of the (a) cationic polymer and the (b) non-polymeric acid having two or more pKa values or a salt thereof can be very stable.

The pH of the composition according to the present invention may be adjusted by adding at least one alkaline agent and/or at least one acid, other than the (b) non-polymeric acid having two or more pKa values or a salt thereof. The pH of the composition according to the present invention may also be adjusted by adding at least one buffering agent.

### (Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as ornithine; and diamines such as those described in the structure below:

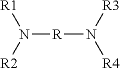

wherein R denotes an alkylene such as propylene optionally substituted by a hydroxyl or a C₁-C₄alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

### (Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as citric acid, lactic acid, sulfuric acid, phosphoric acid and hydrochloric acid (HCl) may be used. Lactic acid may be preferable.

The acid(s) may be used in a total amount of from 0.01% to 15% by weight, preferably from 0.02% to 10% by weight, more preferably from 0.03% to 5% by weight, relative to the total weight of the composition, depending on their solubility.

### (Optional Ingredient)

The composition according to the present invention may comprise, in addition to the aforementioned ingredients, optional ingredient(s) typically employed in cosmetics, specifically, organic or inorganic UV filters; surfactants/emulsifiers, hydrophilic or lipophilic thickeners, derived from, for example, synthetic polymers other than the (a) cationic polymer; volatile or non-volatile organic solvents, such as ethanol; amphoteric polymers; nonionic polymers such as beta-glucan; silicones and silicone derivatives other than the (e) oil; natural extracts derived from animals or vegetables other than the (a) cationic polymer or the (d) oil; waxes; and the like, within a range which does not impair the effects of the present invention.

The composition according to the present invention may comprise the above optional additive(s) in an amount of from 0.01% to 30% by weight, preferably from 0.05% to 20% by weight, and more preferably from 0.1% to 10% by weight, relative to the total weight of the composition.

The composition according to the present invention may include a very limited amount of surfactant(s)/emulsifier(s) and/or synthetic thickener(s) and/or organic solvent(s) in view of environmental friendliness.

The amount of the surfactant(s)/emulsifier(s) and/or synthetic thickener(s) and/or organic solvent(s) in the composition according to the present invention may be 1% by weight or less, preferably 0.1% by weight or less, and more preferably 0.01% by weight or less, relative to the total weight of the composition. It is in particular preferable that the composition according to the present invention include no surfactant/emulsifier or synthetic thickener or organic solvent.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

The composition according to the present invention can be prepared by simple or easy mixing with a conventional mixing means such as a stirrer and a homogenizer. Also, heating may not be necessary. Therefore, the process for preparing the composition according to the present invention may be environmentally friendly.

### [Cosmetic Application]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, and more preferably a skin care composition.

### [Form]

The composition according to the present invention comprises a plurality of aqueous phases and a fatty phase, wherein the aqueous phases are dispersed in the fatty phase. Thus, the aqueous phase can function as a dispersed phase or a discontinuous phase, and the fatty phase can function as a continuous phase.

The aqueous phase comprises the (a) cationic polymer(s), the (b) non-polymeric acid(s) having two or more pKa values or salt(s) thereof, and the (c) water. If the composition according to the present invention comprises the (e) anionic polymer(s), the aqueous phase can comprise the (e) anionic polymer(s).

The fatty phase comprises the (d) oil. If the composition according to the present invention comprises the (f) fatty acid and/or the (g) organo-modified clay, the fatty phase can comprise the (f) fatty acid and/or the (g) organo-modified clay.

The amount of the aqueous phases in the composition according to the present invention may be from 30% to 70% by weight, preferably from 35% to 65% by weight, and more preferably from 40% to 60% by weight, relative to the total weight of the composition.

The amount of the fatty phase in the composition according to the present invention may be from 30% to 70% by weight, preferably from 35% to 65% by weight, and more preferably from 40% to 60% by weight, relative to the total weight of the composition

### [Film]

The composition according to the present invention can be used for easily preparing a film.

Thus, the present invention may also relate to a process for preparing a film, preferably a cosmetic film, optionally with a thickness of preferably more than 0.5 μm, more preferably 1.0 μm or more, and even more preferably 1.5 μm or more, comprising:
applying onto a substrate, preferably a keratin substance, more preferably skin, the composition according to the present invention; and
drying the composition.

The upper limit of the thickness of the film according to the present invention is not limited. Thus, for example, the thickness of the film according to the present invention may be 1 mm or less, preferably 500 μm or less, more preferably 300 μm or less, and even more preferably 100 μm or less.

Since the process for preparing a film according to the present invention includes the steps of applying the composition according to the present invention onto a substrate, preferably a keratin substance, and more preferably skin, and of drying the composition, the process according to the present invention does not require any spin coating or spraying, and therefore, it is possible to easily prepare even a relatively thick film. Thus, the process for preparing a film according to present invention can prepare a relatively thick film without any special equipment such as spin coaters and spraying machines.

Even if the film according to the present invention is relatively thick, it is still thin and may be transparent, and therefore, may not be easy to perceive. Thus, the film according to the present invention can be used preferably as a cosmetic film.

If the substrate is not a keratin substance such as skin, the composition according to the present invention may be applied onto a substrate made from any material other than keratin. The materials of the non-keratinous substrate are not limited. Two or more materials may be used in combination. Thus, a single type of material or a combination of different types of materials may be used. In any event, it is preferable that the substrate be flexible or elastic.

If the substrate is not a keratin substance, it is preferable that the substrate be water-soluble, because it is possible to leave the film according to the present invention by washing the substrate with water. As examples of the water-soluble materials, mention may be made of poly(meth) acrylic acids, polyethyleneglycols, polyacrylamides, polyvinylalcohol (PVA), starch, celluloseacetates, and the like. PVA is preferable.

If the non-keratinous substrate is in the form of a sheet, it may have a thickness of more than that of the film according to the present invention, in order to ease the handling of the film attached to the substrate sheet. The thickness of the non-keratinous substrate sheet is not limited, but may be from 1 m to 5 mm, preferably from 10 μm to 1 mm, and more preferably from 50 to 500 μm.

It is more preferable that the film according to the present invention be releasable from the non-keratinous substrate. The mode of release is not limited. Therefore, the film according to the present invention may be peeled from the non-keratinous substrate, or released by the dissolution of the substrate sheet into a solvent such as water.

The present invention may also relate to:
(1) A film, preferably a cosmetic film, optionally with a thickness of preferably more than 0.5 μm, more preferably 1.0 m or more, and even more preferably 1.5 m or more, prepared by a process comprising:
   applying onto a substrate, preferably a keratin substance, and more preferably skin, the composition according to the present invention; and
   drying the composition,
and
(2) A film, preferably a cosmetic film, optionally with a thickness of preferably more than 0.5 μm, more preferably 1.0 μm or more, and even more preferably 1.5 μm or more, comprising:
   (a) at least one cationic polymer selected from the group consisting of chitosans, polylysines, and mixtures thereof, preferably from chitosans;
   (b) at least one non-polymeric acid having two or more pKa values or a salt thereof; and
   (d) at least one oil.

The film may also include (e) at least one anionic polymer and/or (f) at least one fatty acid and/or (g) at least one organo-modified clay.

The above explanations regarding the (a) cationic polymer, the (b) non-polymeric acid having two or more pKa values or a salt thereof, and the (d) oil, as well as optional ingredients such as the (e) anionic polymer, the (f) fatty acid, and the (g) organo-modified clay, can apply to those in the above films (1) and (2).

The film thus obtained above can be self-standing. The term “self-standing” here means that the film can be in the form of a sheet and can be handled as an independent sheet without the assistance of a substrate or support. Thus, the term “self-standing” may have the same meaning as “self-supporting”.

It is preferable that the film according to the present invention be hydrophobic.

The term “hydrophobic” in the present specification means that the solubility of the film in water (preferably with a volume of 1 liter) at from 20 to 40° C., preferably from 25 to 40° C., and more preferably from 30 to 40° C. is less than 10% by weight, preferably less than 5% by weight, more preferably less than 1% by weight, and even more preferably less than 0.1% by weight, relative to the total weight of the film. It is most preferable that the film is not soluble in water.

If the film according to the present invention is hydrophobic, the film can have water-resistant properties, and therefore, it can remain on a keratin substance such as skin even if the surface of the keratin substance is wet due to, for example, sweat and rain. Thus, when the film according to the present invention provides any cosmetic effect, the cosmetic effect can last a long time.

On the other hand, the film according to the present invention can be easily removed from a keratin substance such as skin under alkaline conditions such as a pH of from 8 to 12, preferably from 9 to 11. Therefore, the film according to the present invention is difficult to remove with water, while it can be easily removed with a soap which can provide such alkaline conditions.

The film according to the present invention may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself or the metabolism of the microorganisms which may be present in the living body. Also, the biodegradable polymer can be degraded by hydrolysis.

If the film according to the present invention includes a biocompatible and/or biodegradable polymer, it may be less irritable or not irritable to the skin and/or it may not contaminate environments.

In fact, the film according to the present invention can include (a) at least one cationic polymer selected from chitosans and/or polylysines both of which are biodegradable polymers. Therefore, the film according to the present invention can be environmentally-friendly.

In addition, due to the use of a biocompatible and/or biodegradable polymer, the cosmetic sheet according to the present invention may adhere well to the skin.

The film according to the present invention can be used for cosmetic treatments of a keratin substance, preferably skin, in particular the face. The film according to the present invention can be in any shape or form. For example, it can be used as a full-face mask sheet, or a patch for a part of the face such as the cheek, nose, and around the eyes.

### [Cosmetic Process and Use]

The present invention also relates to:
a cosmetic process for a keratin substance such as skin, comprising: applying to the keratin substance the composition according to the present invention; and drying the composition to form a cosmetic film on the keratin substance; or
a use of the composition according to the present invention for the preparation of a cosmetic film on a keratin substance such as skin.

The cosmetic process here means a non-therapeutic cosmetic method for caring for and/or making up the surface of a keratin substance such as skin.

In both the above process and use, the above cosmetic film is resistant to water with a pH of 7 or less, and is removable with water with a pH of more than 7, preferably 8 or more, and more preferably 9 or more.

In other words, the above cosmetic film can be water-resistant under neutral or acidic conditions such as a pH of 7 or less, preferably in a range of 6 or more and 7 or less, and more preferably in a range of 5 or more and 7 or less, while the above cosmetic film can be removed under alkaline conditions such as a pH of more than 7, preferably 8 or more, and more preferably 9 or more. The upper limit of the pH is preferably 13, more preferably 12, and even more preferably 11.

Accordingly, the above cosmetic film can be water-resistant, and therefore, it can remain on a keratin substance such as skin even if the surface of the keratin substance is wet due to, for example, sweat and rain. On the other hand, the above cosmetic film can be easily removed from a keratin substance such as skin under alkaline conditions. Therefore, the film according to the present invention is difficult to remove with water, while it can be easily removed with a soap which can provide alkaline conditions.

If the above cosmetic film includes a UV filter which may be present in the composition according to the present invention, the above cosmetic film can protect a keratin substance such as skin from UV rays, thereby limiting the darkening of the skin, improving the color and uniformity of the complexion, and/or treating aging of the skin.

Furthermore, the above cosmetic film may have cosmetic effects such as capturing sebum, matting the appearance of a keratin substrate such as skin, absorbing or adsorbing malodour, and/or protecting the keratin substance from, for example, dirt or pollutant, even if the cosmetic film does not include any cosmetic active ingredient.

In addition, the above cosmetic film may immediately change or modify the appearance of the skin by changing light reflection on the skin and the like, even if the cosmetic film does not include any cosmetic active ingredient. Therefore, it may be possible for the above cosmetic film to conceal skin defects such as pores or wrinkles. Further, the above cosmetic film may immediately change or modify the feel to the touch of the skin by changing the surface roughness on the skin and the like. Furthermore, the above cosmetic film may immediately protect the skin by covering the surface of the skin and shielding the skin, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient other than the (d) oil, the cosmetic film can have cosmetic effects provided by the additional cosmetic active ingredient(s). For example, if the cosmetic film includes at least one cosmetic active ingredient selected from anti-aging agents, anti-sebum agents, deodorant agents, anti-perspirant agents, whitening agents and a mixture thereof, the cosmetic film can treat the aging of the skin, absorbing sebum on the skin, controlling odors on the skin, controlling perspiration on the skin, and/or whitening of the skin.

It is also possible to apply a makeup cosmetic composition onto the cosmetic film or sheet according to the present invention after it has been applied onto the skin.

The present invention may also relate to a use of
(a) at least one cationic polymer selected from the group consisting of chitosans, polylysines, and mixtures thereof, preferably from chitosans, and
(b) at least one non-polymeric acid having two or more pKa values or a salt thereof, in a composition, comprising:
a plurality of aqueous phases comprising:
(c) water,
and
a fatty phase comprising:
(d) at least one oil,
wherein
the aqueous phases aqueous phases are dispersed in the fatty phase,
in order to provide a keratin substance such as skin with good hydration.

The use according to the present invention can also provide a keratin substance such as skin with anti-greasiness and/or anti-color transfer and/or anti-stickiness.

The composition may also include (e) at least one anionic polymer and/or (f) at least one fatty acid and/or (g) at least one organo-modified clay. If the composition includes (f) at least one fatty acid, the anti-color transfer can be further enhanced.

The above explanations regarding the (a) cationic polymer, the (b) non-polymeric acid having two or more pKa values or a salt thereof, the (c) water and the (d) oil, as well as optional ingredients such as the (e) anionic polymer, the (f) fatty acid, and the (g) organo-modified clay can apply to those in the above use.

### EXAMPLES

The present invention will be described in a more detailed manner by way of examples. However, they should not be construed as limiting the scope of the present invention.

### Examples 1-6 and Comparative Examples 1-5

### [Preparations]

Each of the compositions according to Examples 1-6 and Comparative Examples 1-5 was prepared by mixing the ingredients shown in Tables 1-4. The numerical values for the amounts of the ingredients in Tables 1-4 are all based on “% by weight” as raw materials.

The details of the preparation are as follows.

First, all the ingredients for the oil phase were mixed well. On the other hand, the aqueous phase was separately prepared by the following method: a polyanion (sodium hyaluronate, and, if present, sodium carboxymethylcellulose) was first dissolved in water. Then, a polycation (polylysine or chitosan) and a crosslinker (phytic acid) and was added to make a polyion complex gel particle (PGP). After all the other ingredients for the aqueous phase were added, the aqueous phase was added to the oil phase and emulsified to form a W/O type composition. For Example 3-5 and Comparative Examples 2-3, ethanol was added after the emulsification.

TABLE 1

| | | | | Comp. |
|---|---|---|---|---|
| | | Ex. 1 | Ex. 2 | Ex. 1 |
| FP | PEG-9 POLYDIMETHYLSILOXYETHYL | 3.5 | 3.5 | 3.5 |
| | DIMETHICONE | | | |
| | SORBITAN ISOSTEARATE | 0.5 | 0.5 | 0.5 |
| | CETYL ETHYLHEXANOATE | 12 | 12 | 12 |
| | HOMOSALATE | 3 | 3 | 3 |
| | DIMETHICONE | qsp 100 | qsp 100 | qsp 100 |
| | ISOHEXADECANE | 7.5 | 7.5 | 7.5 |
| | OLEIC ACID | — | 1.7 | — |
| | DEXTRIN PALMITATE | 2 | 2 | 2 |
| | DIMETHICONE (and) DIMETHICONE/VINYL | 2 | 2 | 2 |
| | DIMETHICONE CROSSPOLYMER | | | |
| | SURFACE-TREATED TITANIUM DIOXIDE | 5.5 | 5.5 | 5.5 |
| AP | WATER | 39.68 | 39.68 | 39.68 |
| | MAGNESIUM SULFATE | — | — | 0.7 |
| | GLYCERIN | 6 | 6 | 6 |
| | PHENOXYETHANOL | 0.7 | 0.7 | 0.7 |
| | BUTYLENE GLYCOL | 9 | 9 | 9 |
| | CELLULOSE GUM | — | — | 0.5 |
| | SODIUM HYALURONATE | 0.53 | 0.53 | 0.53 |
| | POLYLYSINE (25 wt % Aqueous Solution) | 1.06 | 1.06 | — |
| | PHYTIC ACID (50 wt % Aqueous Solution) | 0.27 | 0.27 | — |

| | | | |
|---|---|---|---|
| SCH Value (Before Application) (a) | 18 | 16 | 16 |
| SCH Value (4 Hours After Application) (b) | 46 | 41 | 37 |
| Hydration ((b) − (a)) | Good | Good | Control |
| Anti-Greasiness | Good | Good | Control |
| Anti-Mask Transfer | Good | Very | Control |
| | | Good | |

FP: Fatty Phase
AP: Aqueous Phase

TABLE 2

| | | | | Comp. |
|---|---|---|---|---|
| | | Ex. 3 | Ex. 4 | Ex. 2 |
| FP | DIISOPROPYL SEBACATE | 6.25 | 6.25 | 6.25 |
| | DIMETHICONE | qsp 100 | qsp 100 | qsp 100 |
| | ISODODECANE | 10 | 10 | 10 |
| | PEG-10 DIMETHICONE | 5 | 5 | 5 |
| | TOCOPHEROL | 0.1 | 0.1 | 0.1 |
| | TRIMETHYLSILOXYSILICATE | 5 | 5 | 5 |
| | DISTEARDIMONIUM HECTORITE | 1 | 1 | 1 |
| | SURFACE-TREATED TITANIUM DIOXIDE | 1 | 1 | 1 |
| | SURFACE-TREATED IRON OXIDES | 0.1 | 0.1 | 0.1 |
| | SILICA | 10.5 | 10.5 | 10.5 |
| AP | WATER | 26 | 26.375 | 26 |
| | SODIUM HYALURONATE | 0.25 | 0.25 | — |
| | POLYLYSINE (25 wt % Aqueous Solution) | 0.5 | — | — |
| | CHITOSAN | — | 0.125 | — |
| | PHYIC ACID (50 wt % Aqueous Solution) | 0.14 | 0.14 | — |
| | GLYCERIN | 11 | 11 | 11 |
| | PHENOXYETHANOL | 0.5 | 0.5 | 0.5 |
| | ETHANOL | 5 | 5 | 5 |

| | | | |
|---|---|---|---|
| SCH Value (Before Application) (a) | 12.0 ± 2.3 | 13.1 ± 2.8 | 12.2 ± 2.7 |
| SCH Value (4 Hours After Application) (b) | 36.8 ± 2.1 | 38.3 ± 2.1 | 35.8 ± 2.6 |
| Hydration ((b) − (a)) | Good | Good | Control |

FP: Fatty Phase
AP: Aqueous Phase

TABLE 3

| | | | Comp. |
|---|---|---|---|
| | | Ex. 5 | Ex. 3 |
| FP | DIISOPROPYL SEBACATE | 2 | 2 |
| | DIMETHICONE | qsp 100 | qsp 100 |
| | ISODODECANE | 10 | 10 |
| | PEG-10 DIMETHICONE | 5 | 5 |
| | TOCOPHEROL | 0.1 | 0.1 |
| | TRIMETHYLSILOXYSILICATE | 5 | 5 |
| | DISTEARDIMONIUM HECTORITE | 1 | 1 |
| | SURFACE-TREATED | 1 | 1 |
| | TITANIUM DIOXIDE | | |
| | SURFACE-TREATED | 0.1 | 0.1 |
| | IRON OXIDES | | |
| | SILICA | 10 | 10 |
| AP | WATER | 26 | 26 |
| | SODIUM HYALURONATE | 0.06 | 0.06 |
| | SODIUM | 0.19 | 0.19 |
| | CARBOXYMETHYLCELLULOSE | | |
| | POLYLYSINE (25 wt % | 0.5 | — |
| | Aqueous Solution) | | |
| | PHYTIC ACID (50 wt % | 0.14 | — |
| | Aqueous Solution) | | |
| | BUTYLENE GLYCOL | 5 | 5 |
| | GLYCERIN | 6 | 6 |
| | MAGNESIUM SULFATE | 0.7 | 0.7 |
| | PHENOXYETHANOL | 0.5 | 0.5 |
| | ETHANOL | 5 | 5 |

| | | |
|---|---|---|
| SCH Value (Before Application) (a) | 11.6 ± 1.5 | 12.6 ± 1.4 |
| SCH Value (4 Hours After Application) (b) | 36.4 ± 1.8 | 32.4 ± 5.0 |
| Hydration ((b) − (a)) | Good | Control |

FP: Fatty Phase
AP: Aqueous Phase

TABLE 4

| | | | Comp. | Comp. |
|---|---|---|---|---|
| | | Ex. 6 | Ex. 4 | Ex. 5 |
| FP | DIISOPROPYL SEBACATE | 2 | 2 | 2 |
| | DIMETHICONE | qsp 100 | qsp 100 | qsp 100 |
| | ISODODECANE | 10 | 10 | 10 |
| | PEG-10 DIMETHICONE | 5 | 5 | 5 |
| | TOCOPHEROL | 0.1 | 0.1 | 0.1 |
| | TRIMETHYLSILOXYSILICATE | 5 | 5 | 5 |
| | DISTEARDIMONIUM HECTORITE | 1 | 1 | 1 |
| | SURFACE-TREATED TITANIUM DIOXIDE | 1 | 1 | 1 |
| | SURFACE-TREATED IRON OXIDES | 0.1 | 0.1 | 0.1 |
| | SILICA | 10 | 10 | 10 |
| AP | WATER | 22 | 22.64 | 22.89 |
| | SODIUM HYALURONATE | 0.25 | 0.25 | — |
| | POLYLYSINE (25 wt % Aqueous Solution) | 0.5 | — | — |
| | PHYTIC ACID (50 wt % Aqueous Solution) | 0.14 | — | — |
| | BUTYLENE GLYCOL | 5 | 5 | 5 |
| | GLYCERIN | 10 | 10 | 10 |
| | MAGNESIUM SULFATE | 0.7 | 0.7 | 0.7 |
| | PHENOXYETHANOL | 0.5 | 0.5 | 0.5 |
| | ETHANOL | 5 | 5 | 5 |

| | | | |
|---|---|---|---|
| SCH Value (Before Application) (a) | 11.5 ± 3.0 | 9.5 ± 2.7 | — |
| SCH Value (4 Hours After Application) (b) | 35.0 ± 3.7 | 30.4 ± 4.4 | — |
| Hydration ((b) − (a)) | Good | Control | — |
| Evaluation of Stickiness | Good | Poor | Control |

FP: Fatty Phase
AP: Aqueous Phase

### [Evaluations]

### (Hydration)

Hydration was evaluated by comparing the stratum corneum hydration (SCH) values of a bare skin before application and after 4 hours application of each of the compositions according to Examples 1-6 and Comparative Examples 1-4.

As an evaluation device, a GP SKIN device (GPOWER Inc.) was used to measure SCH.

First, the (a) SCH value of the forearm of panelists was measured. Then, 50 mg of each of the compositions according to Examples 1-6 and Comparative Examples 1-4 was applied on the forearm (5×5 cm). After 4 hours, the composition was wiped off, and the (b) SCH value of the forearm was measured again. The difference of the SCH values ((b)-(a)) before the application and 4 hours after the application was calculated. The difference which indicates hydration was evaluated in accordance with the following criteria.
Good: The difference is larger than that of the control
Poor: Same as the control

Regarding the control, please refer to Tables 1-4.

The measured values of the SCH values and the results of the above evaluation are shown in Tables 1-4.

### (Anti-Greasiness and Anti-Mask Transfer)

200 mg of the composition according to Example 1 or 2 was applied on half of the face of two panelists. 200 mg of the composition according to Comparative Example 1 was also applied, as a control, on the other half of the face of the two panellists. Then, a conventional foundation was applied onto the face and a non-woven mask was put on the face.

After 8 hours, the greasiness of skin was visually evaluated by checking the shine level on the face skin by the experts. Mask-transferring was also visually evaluated by the color transfer to the mask.
Very Good: Much less greasiness/mask transferring than the control
Good: Less greasiness/mask transferring than the control
Poor: Same as the control

The results are shown in the lines of “Anti-Greasiness” and “Anti-Mask Transfer” in Table 1.

### (Evaluation of Stickiness)

Each of the compositions according to Example 6, Comparative Example 4 or Comparative Example 5 was applied on the skin of 3 panelists. The composition according to Comparative Example 5 was used as a control. The stickiness of the applied skin was evaluated by the 3 panelists in accordance with the following criteria.
Good: Less sticky than the control
Poor: Same as the control
Very Poor: Worse than the control

The results are shown in Table 4.

### SUMMARY

According to Table 1, it can be found by comparing Example 1 with Comparative Example 1 that the former showed better hydration, anti-greasiness, and anti-mask transfer by having the polycation, the polyanion, and the crosslinker, than the latter. Furthermore, by adding a fatty acid (oleic acid) (cf. Example 2), the anti-mask transfer property was enhanced.

According to Table 2, it can be found by comparing Example 3 or 4 with Comparative Example 2 that the former showed better hydration by having the polycation, the polyanion, and the crosslinker, than the latter. When using chitosan instead of polylysine as the polycation (cf. Example 4), the hydration was further enhanced.

According to Table 3, it can be found by comparing Example 5 with Comparative Example 3 that the former showed better hydration by having the polycation (polylysine) and the crosslinker (phytic acid), in addition to the polyanions (sodium hyaluronate and sodium CMC), than the latter including only the polyanions.

According to Table 4, it can be found by comparing Example 6 with Comparative Example 4 that the former showed better hydration by having the polycation (polylysine) and the crosslinker (phytic acid), in addition to the polyanion (sodium hyaluronate), than the latter including only the polyanion.

In addition, by comparing Comparative Example 4 with Comparative Example 5 in Table 4, it can be found that stickiness became worse by only having the polyanion (sodium hyaluronate). However, by adding the polycation and the crosslinker (cf. Example 6), stickiness was reduced. It can be deduced that stickiness mainly coming from glycerin was covered by having a glyceryl gel network which consists of the polycation, the crosslinker, the polyanion, and glycerin. However, only the polyanion cannot create any glycerin gel network, which even enhances stickiness.

1. A composition, comprising
a plurality of aqueous phases comprising:
(a) at least one cationic polymer;
(b) at least one non-polymeric acid having two or more pKa values or a salt thereof; and
(c) water,
and
a fatty phase comprising:
(d) at least one oil,
wherein
the aqueous phases are dispersed in the fatty phase, and the (a) cationic polymer is selected from the group consisting of chitosans, polylysines and mixtures thereof.

2. The composition according to claim 1, wherein the amount of the (a) cationic polymer(s) in the composition is from 0.01% to 15% by weight, relative to the total weight of the composition.

3. The composition according to claim 1, wherein the (b) non-polymeric acid having two or more pKa values or a salt thereof is an organic acid or a salt thereof.

4. The composition according to claim 1, wherein the amount of the (b) non-polymeric acid(s) having two or more pKa values or salt(s) thereof in the composition is from 0.001% to 10% by weight, relative to the total weight of the composition.

5. The composition according to claim 1, wherein the amount of the (c) water in the composition is from 10% to 50% by weight, relative to the total weight of the composition.

6. The composition according to claim 1, wherein the amount of the (d) oil(s) in the composition is from 10% to 50% by weight, relative to the total weight of the composition.

7. The composition according to claim 1, wherein the aqueous phase further comprises (e) at least one anionic polymer.

8. The composition according to claim 7, wherein the (e) anionic polymer is selected from the group consisting of polysaccharides, anionic (co)polyaminoacids, (co)poly(meth)acrylic acids, (co)polyamic acids, (co)polystyrene sulfonate, (co)poly(vinyl sulfates), dextran sulfate, chondroitin sulfate, (co)polymaleic acids, polyfumaric acids, maleic acid (co)polymers, and salts thereof.

9. The composition according to claim 7, wherein the amount of the (e) anionic polymer(s) in the composition is from 0.001% to 15% by weight, relative to the total weight of the composition.

10. The composition according to claim 1, wherein the fatty phase further comprises (f) at least one fatty acid.

11. The composition according to claim 10, wherein the amount of the (f) fatty acid(s) in the composition is from 0.01% to 15% by weight, relative to the total weight of the composition.

12. The composition according to claim 1, wherein the fatty phase further comprises (g) at least one organic-modified clay mineral.

13. The composition according to claim 1, wherein the amount of the aqueous phases in the composition is from 30% to 70% by weight, relative to the total weight of the composition.

14. The composition according to claim 1, wherein the amount of the fatty phase in the composition is from 30% to 70% by weight, relative to the total weight of the composition.

15. A cosmetic process for a keratin substance, comprising:
applying to the keratin substance the composition according to claim 1; and
drying the composition to form a cosmetic film on the keratin substance.
